(19) United States
(12) Patent Application Publication
(10) Pub. No.: US 2024/0321452 A1
(43) Pub. Date: Sep. 26, 2024

(54) DEVICE AND METHOD FOR PREDICTING AUTISM SPECTRUM DISORDER IN INFANTS AND YOUNG CHILDREN ON BASIS OF DEEP LEARNING

(71) Applicant: Gwangju Institute of Science and Technology, Gwangju (KR)

(72) Inventors: Hong Kook KIM, Gwangju (KR); Jung Hyuk LEE, Gwangju (KR); Geon Woo LEE, Gwangju (KR)

(73) Assignee: Gwangju Institute of Science and Technology, Gwangju (KR)

(21) Appl. No.: 18/579,519

(22) PCT Filed: Aug. 9, 2022
(86) PCT No.: PCT/KR2022/011812

§ 371 Date: Jan. 16, 2024

Related U.S. Application Data
(60) Provisional application No. 63/249,587, filed on Sep. 29, 2021.

(30) Foreign Application Priority Data
Jul. 26, 2022 (KR) 10-2022-0092428

(51) Int. Cl. G16H 50/20; A61B 5/00

(52) U.S. Cl. CPC G16H 50/20; A61B 5/4803

(57) ABSTRACT

The present invention relates to disorder spectrum diagnosis technology, and more particularly, to a device and method for predicting autism spectrum disorder in infants and young children on the basis of deep learning by using auto-encoder feature representation, wherein autism spectrum disorder can be identified from the speech of infants and young children by using auto-encoder feature representation.

### TECHNICAL FIELD

The present invention relates to disorder spectrum diagnosis technology, and more particularly, to a device and method for predicting autism spectrum disorder in infants and young children on the basis of deep learning by using auto-encoder feature representation, wherein autism spectrum disorder can be identified from the speech of infants and young children by using auto-encoder feature representation.

### BACKGROUND ART

Autism has various types and degrees depending on its features, and thus, it is called a spectrum.

Although diagnostic instruments have been developed and validated on the basis of the accuracy in distinguishing children with autism spectrum disorder (ASD) from children with typical development (TD), the stability of procedure may be disrupted due to time constraints and clinician's subjectivity.

According to the diagnostic and statistical manual of mental disorders, fifth edition (DSM-5), the autism spectrum disorder includes several symptoms such as restricted interests or behaviors, delayed language development and impaired social communication and interaction.

A variety of evidence that people with autism spectrum disorder (ASD) are more likely to improve their social competence when they receive early clinical intervention have been obtained through prior studies and thus, early detection of autism spectrum disorder (ASD) features may be said to be a key point in current autism spectrum disorder (ASD) research.

Accordingly, an automatic diagnosis technique for obtaining an objective measurement of autism spectrum disorder (ASD) has been developed and phonetic features have been being reported in various fields of research and furthermore, although considered as a clinician's unique characteristic, several studies utilizing a deep-learning model based on automated distinguishment between children with autism spectrum disorder (ASD) and children with typical development (TD) have also shown promising performance.

However, there still exist difficulties such as the lack of organized data due to the feature of data, the complexity of analysis, the low accessibility to diagnosis, the necessity to secure anonymity and the like. The quality of prior studies based on a variety of acoustic features has demonstrated the effectiveness of the acoustic features and classification algorithms for detecting abnormalities in children's speeches in a group of children with autism spectrum disorder (ASD) distinguished from a group of children with typical development (TD), however, the complexity and uniqueness relationship between the features remain the same and thus, is uncertain until a large amount of data is accumulated. In order to solve these difficulties, the present invention proposes a method of improve the detection of the autism spectrum in infants and young children by acquiring speech feature representations based on an auto-encoder (AE).

### DETAILED DESCRIPTION OF THE INVENTION

### Technical Problem

The present invention relates to the detection of autism spectrum disorder, and provides a device and method for predicting autism spectrum disorder in infants and young children on the basis of deep learning that can predict autism spectrum disorder by adding an auto-encoder for extracting the features of speech data of infants and young children using the features of delayed language development.

### Technical Solution

According to one aspect of the present invention, a deep learning-based device for predicting autism spectrum disorder in infants and young children is provided.

A deep learning-based device for predicting autism spectrum disorder in infants and young children according to an embodiment of the present invention may comprise an input unit for segmenting speech data, a first extraction unit for extracting speech features for classifying autism spectrum disorder (ASD), a second extraction unit for extracting auto-encoder-based speech features, and a classification unit for classifying the autism spectrum disorder using the speech features.

According to other aspect of the present invention, a deep learning-based method for predicting autism spectrum disorder in infants and young children, and a computer program for executing the same are provided.

A deep learning-based method for predicting autism spectrum disorder in infants and young children according to an embodiment of the present invention and a computer program executing the same may comprises the steps of receiving and segmenting speech data, extracting speech features from the speech data, and extracting feature values using an auto-encoder.

### Effects of the Invention

According to one aspect of the present invention, it is possible to increase the reliability of classification of autism spectrum disorder by adding an auto-encoder in extracting the feature using speech in the early development of autistic children.

### BRIEF DESCRIPTION OF THE DRAWINGS

FIGS. 1 and 2 are diagrams illustrating a deep learning-based device for predicting autism spectrum disorder in infants and young children according to an embodiment of the present invention.

FIG. 3 is an example diagram illustrating a joint optimization learning model of a deep learning-based device for predicting autism spectrum disorder in infants and young children according to an embodiment of the present invention.

FIG. 4 is a diagram showing a deep learning-based method for predicting autism spectrum disorder in infants and young children according to an embodiment of the present invention.

### BEST MODE FOR CARRYING OUT THE INVENTION

The present invention can make various changes and have various embodiments and specific embodiments will be illustrated in the drawings and described in detail through detailed description. However, it is not intended to limit the present invention to such specific embodiments and should be understood to encompass all changes, equivalents, and substitutes falling within in the spirit and technical scope of the present invention. In describing the present invention, if it is determined that a detailed description of related known technologies may unnecessarily obscure the gist of the present invention, the detailed description will be omitted. Additionally, as used in the present specification and claims, the singular representations should generally be construed to mean “one or more” unless otherwise specified.

Hereinafter, preferred embodiments of the present invention will be described in detail with reference to the accompanying drawings. In the description with reference to the accompanying drawings, identical or corresponding components will be assigned the same reference numerals and redundant description thereof will be omitted.

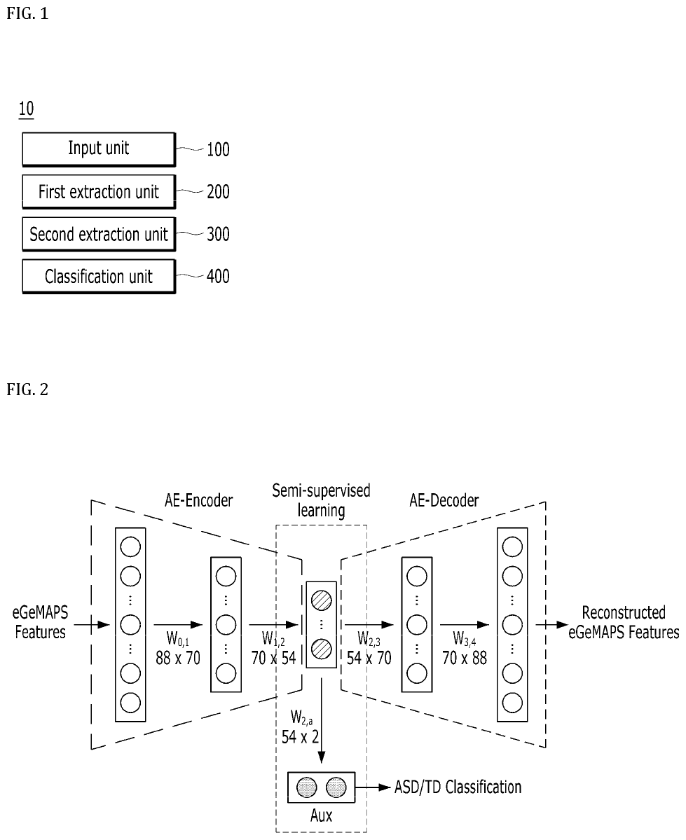

FIGS. 1 and 2 are diagrams illustrating a deep learning-based device for predicting autism spectrum disorder in infants and young children according to an embodiment of the present invention.

Referring to FIG. 1, the deep learning-based device 10 for predicting autism spectrum disorder in infants and young children may include an input unit 100, a first extraction unit 200, a second extraction unit 300, and a classification unit 400.

The input unit 100 divides and uses only the infants and young children speeches from the speech data.

In order to analyze the speech feature, the input unit 100 may divide the speech data into audio segments including only the infant and young children's speeches that do not overlap with other sounds and other people's speeches.

The first extraction unit 200 can extract speech features for classifying the autism spectrum disorder (ASD) from the infant and young children's speech data. For example, the first extraction unit 200 may use the extended Geneva Minimalistic Acoustic Parameter Set (eGeMAPS) for extraction of speech features in order to obtain a set of effective features having the quality for the speech data. The Geneva Minimalistic Acoustic Parameter Set (GeMAPS) is a popular set of functions that provides minimalistic speech features commonly used in automatic speech analysis, rather than a large brute-force parameter set. The eGe-MAPS, which is the extended version thereof, includes 88 speech functions that have been fully utilized in the present invention.

The first extraction unit 200 downsamples and downmixes each recorded audio data set stored as a 48 KHz stereo file into a 16 kHz mono audio file with consideration for usability and resolution in mel-frequency cepstral coefficients (MFCCs).

In order to extract speech features for classification of the ASD, the first extraction unit 200 divides each infant and young children's speeches into 25 ms frames with 10 ms overlap between frames. Then, the input unit 100 can extract various speech features for each frame with open source speech and acoustic analysis using the OpenSMILE toolkit. For example, the first extraction unit 200 can extract various features of 88 eGeMAPS for each frame. The input unit 100 can normalize the extracted features by average and standard deviation.

The first extraction unit 200 can obtain normalization scaling by normalizing factors of training data set and modify it.

The first extraction unit 200 may group the normalized features into each five frames with consideration for the time-related features of the speech data.

The first extraction unit 200 can extract speech features for classification of the ASD from the infant and young children's speech data. For example, the first extraction unit 200 can extract eGeMAPS features from infants and young children's speech data.

The second extraction unit 300 can use an auto-encoder (AE) model to extract features for diagnosing the ASD. That is, the second extraction unit 300 can use an auto-encoder (AE)-based speech feature extraction model.

FIG. 2 is an example of an auto-encoder training model.

The AE model converts input parameters into a latent representation using a hidden layer.

Assuming that the input of the AE model is x∈R^{d}, the latent representation z∈R^{d}′ and the reconstructed input y∈R^{d} can be obtained by applying a non-linear activation function f to the weighted sum of z using the weight matrix W∈R^{dxd′} and the bias vector b∈R^{d′} as shown in Equation 1 below:

$$z = f ( {W}^{T} x + b ) \qquad Equation 1$$

$$y = f ( {W}^{T} z + {b}^{′} ) ,$$

where T is the transpose operator of a matrix.

When the latent dimension d′<d, the output of the latent layer is considered as compressed meaningful values extracted from the input and can be referred to as the bottleneck feature.

Referring to FIG. 2, the AE training model may be composed of input, hidden, latent, hidden and output layers. For example, each layer may be constructed to have dimensions of 88, 70, 54, 70 and 88 nodes, respectively, as a fully connected (FC) layer.

The AE training model has an encoder (AE-Encoder, 310) and a decoder (AE-Decoder, 320) structured symmetrically around the latent layer. The encoder (AE-Encoder, 310) may be composed from the input layer to the latent layer, and the decoder (AE-Decoder, 320) may include from the bottleneck point to the output layer. The second extraction unit 300 is reduced compared to the input layer and may include a latent layer with compressed feature dimensions. The second extraction unit 300 can reconstruct the speech features using the speech features extracted by the first extraction unit 200 as an input value. The second extraction unit 300 can reconstruct and extract the speech features using a deep learning model that uses the speech features extracted by the first extraction unit 200 as an input value.

The second extraction unit 300 uses the AE model, and thus, it can convert the speech features into a latent representation that can better represent distinguishable features of the data, through a feature value embedding. The second extraction unit 300 can improve the embedding performance through semi-supervised learning by applying a multi-task learning which inputs latent representation values and outputs autism spectrum disorder (ASD)/typical development (TD) test results.

The second extraction unit 300 may use the normalized speech features of the first extraction unit 200 as an input.

The second extraction unit 300 may add an auxiliary output (AUX) that classifies autism spectrum disorder (ASD) and typical development (TD) into binary categorical targets through the semi-supervised learning.

The second extraction unit 300 can calculate classification results based on the reconstructed speech feature and auxiliary output as shown in Equation 2 below:

$${z}_{i} = f ( {W}_{i - 1} , {z}_{i - 1} + {b}_{i - 1 , i} ) \qquad Equation 2$$

$$where {z}_{1} = f ( {W}_{0 , 1} + {b}_{0 , 1} ) , and$$

$${y}_{r e c} = {W}_{3 , 4} {z}_{3} + {b}_{3 , 4}$$

$${y}_{aux} = \partial ( {W}_{2 , a} {z}_{2} + {b}_{2 , a} ) ,$$

where y_{rec} represents the reconstructed speech feature,
yₐᵤₓ is the classification result by auxiliary output, f is an activation function, and ∂ is a softmax activation.

The second extraction unit 300 measures the loss of the reconstruction error using the average absolute error, while the second extraction unit 300 measures the loss of the classification result of the auxiliary output using a binary cross-entropy loss function.

The second extraction unit 300 can combine the reconstruction error loss and the loss of the classification result of the auxiliary output into reasonable hyper parameter and optimize them at the same time.

The premise loss equation is as Equation 3 below:

$${L}_{r e c o n} = \frac{1}{N} {\sum }_{i = 1}^{N} \vert {y}_{{i}_{yec}} - {y}_{{i}_{gy}} \vert \qquad Equation 3$$

$${L}_{aux} = - {y}_{{1}_{gy}} log ( {y}_{{1}_{aux}} ) - ( 1 - t ) {y}_{{1}_{gy}} log ( {y}_{{1}_{aux}} )$$

$${L}_{total} = {L}_{r e c o n} + α {L}_{aux} ,$$

where

L_{recon} is a loss of reconstruction error

Lₐᵤₓ, is a loss of classification result loss of auxiliary output using binary cross-entropy loss function.

Lₜₒₜₐₗ is the total loss.

The classification unit 400 may construct a deep-learning learning model for distinguishing the ASD using the latent representation output from the second extraction unit 300 as input. For example, the classification unit 400 may be constructed by a deep-learning learning model such as a bidirectional LSTM (BLSTM) which uses, as an input, the latent representation encoded and output by the second extraction unit 300 using the grouped speech features extracted from the first extraction unit 200 as input values and which is directed at classification labels for the autism spectrum infant and young children and typical development infant and young children.

The classification unit 400 may apply a batch normalization, a rectangle linear unit (ReLU) activation and a dropout to each layer except the output layer, and use an adaptive momentum (ADAM) optimization. The classification unit 400 can perform a control by stopping training early to minimize validation error within 100 epochs and storing the best BLSTM learning model to improve validation loss for each epoch.

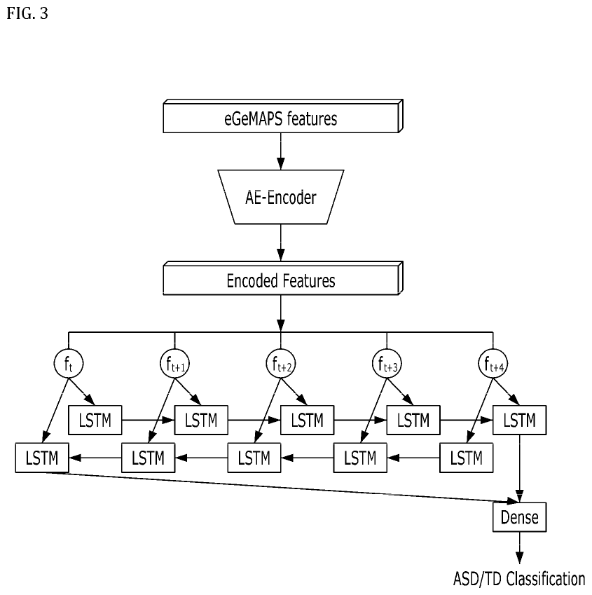

FIG. 3 is a diagram illustrating the joint optimization learning model of the auto-encoder and BLSTM of the deep learning-based device for predicting autism spectrum disorder in infants and young children according to an embodiment of the present invention.

Referring to FIG. 3, the joint optimization model of the auto-encoder and BLSTM can use, as input values of deep learning-based classifier model, the speech features reconstructed by the second extraction unit 300 using the grouped speech features extracted by the first extraction unit 200 as input values. For example, the deep learning-based classifier model may include a BLSTM learning model.

According to the embodiment, the deep learning-based device 10 for predicting autism spectrum disorder in infants and young children can construct a joint optimization model using the auto-encoder and BLSTM by constructing the feature extraction part using the encoder 310 of the second extraction unit 300 on a trained BLSTM learning model.

The deep learning-based device 10 for predicting autism spectrum disorder in infants and young children can distinguish autism spectrum disorder and typical development using a joint optimization model of auto-encoder and BLSTM.

### Example of Experiment

To compare performance, an experiment was conducted using the BLSTM model, which has a different type of input feature from a support vector machine (SVM).

Table 1 is the average performance table of five verification splits for the BLSTM using the SVM, the BLSTM with 88 or 54 eGeMAPS features and the auto-encoder.

TABLE 1

| | Models | | | | | | | |
|---|---|---|---|---|---|---|---|---|
| | | | BLSTM | | BLSTM | | BLSTM | |
| | SVM | | (eGeMAPS-54) | | (eGeMAPS-88) | | (AE-Encoded) | |
| Predicted To | ASD | TD | ASD | TD | ASD | TD | ASD | TD |
| ASD | 62 | 18 | 170 | 103 | 196 | 99 | 215 | 98 |
| TD | 413 | 632 | 305 | 547 | 279 | 551 | 260 | 552 |

| | | | | |
|---|---|---|---|---|
| Accuracy | 0.6178 | 0.6373 | 0.6640 | 0.6818 |
| Precision | 0.1305 | 0.3579 | 0.4126 | 0.4526 |
| Recall | 0.7750 | 0.6227 | 0.6644 | 0.6869 |
| F1 score | 0.2234 | 0.4545 | 0.5091 | 0.5457 |
| UAR | 0.5514 | 0.5997 | 0.6302 | 0.6509 |

UAR, unweighted average recall.

The models in the BLSTM labels in Table 1 are features through BLSTM model learning in which eGeMAPS-54 represents 54 features selected by the Mann-Whitney U test, eGeMAPS-88 represents 88 features of eGeMAPS, and AE-Encoded is a joint optimization model using the auto-encoder and BLSTM.

The performance of each method was evaluated through five-fold cross-validation in which 95 average autism spectrum disorder speeches and 130 average developmental speeches are proportionally distributed over five speech cases for generalized estimation of unfocused speech data.

In the classification stage during the experiment, one speech has been processed in a frame-wise manner, the softmax output has been converted to class index 0 and 1, and if the average class index of the frame is 0.5 or more, the speech has been considered to be a speech of children with ASD.

Performance has been scored using a weighted average recall (UAR) and a weighted average recall (WAR) selected from the INTERSPEECH 2009 emotion challenge, which takes imbalanced classes into account as well as existing measures.

As shown in Table 1, in the experiment, the SVM model has shown very low precision and extremely biased toward the typical development (TD) class. Although the BLSTM (eGeMAPS-88) model has shown significant quality in terms of classifying children with autism spectrum disorder and (ASD) children with typical development (TD), it can be seen that the performance of the BLSTM (AE-Encoded) model has been improved in terms of accurately classifying children with autism spectrum disorder (ASD), compared to the BLSTM (eGeMAPS-88). The BLSTM (eGeMAPS-54) model had lower quality compared to BLSTM (eGeMAPS-88) and yielded results more biased toward children with typical development (TD).

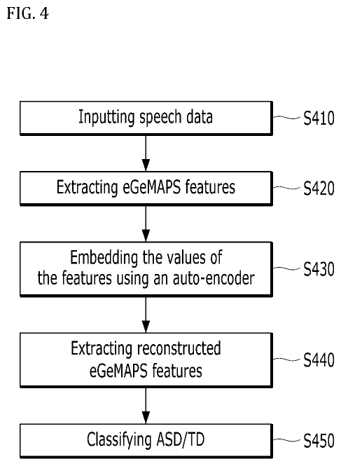

FIG. 4 is a diagram illustrating a deep learning-based method for predicting autism spectrum disorder in infants and young children according to an embodiment of the present invention. Each process described below is a process performed at each stage by each functional unit constituting the deep learning-based device for predicting autism spectrum disorder in infants and young children, but for a concise and clear explanation of the present invention, the subject of performing each step shall be collectively referred to as a deep learning-based device for predicting autism spectrum disorder in infants and young children.

Referring to FIG. 4, in step S410, the deep learning-based device 10 for predicting autism spectrum disorder in infants and young children segments only the speeches of the infant and young children who is the main speaker from the speech data input for autism spectrum disorder classification.

In step S420, the deep learning-based device 10 for predicting autism spectrum disorder in infants and young children extracts speech features from the segmented speech data of the infants and young children. For example, the deep learning-based device 10 for predicting autism spectrum disorder in infants and young children extracts eGeMAPS features.

In step S430, the deep learning-based device 10 for predicting autism spectrum disorder in infants and young children embeds the values of the features using an auto-encoder.

The auto-encoder (AE) model converts input parameters into a latent representation using a hidden layer and then reconstructs the input parameters with latent values.

In step S440, the deep learning-based device 10 for predicting autism spectrum disorder in infants and young children extracts the latent representation through the encoder unit of the auto-encoder.

In step S450, the deep learning-based device 10 for predicting autism spectrum disorder in infants and young children classifies autism spectrum disorder through a deep learning-based classifier model using the latent representation extracted in step S440 as input. For example, the deep learning-based device 10 for predicting autism spectrum disorder in infants and young children can classify autism spectrum disorder based on the BLSTM model.

A confusion can arise in distinguishing between autism spectrum disorder (ASD) and typical development (TD) because speech features include visual features associated with speech feature vocalization and speech.

However, the present invention uses reconstructed eGeMAPS features with a more characteristic distribution compared to eGeMAPS used as an example of speech features. The eGeMAPS feature which has been encoded and reconstructed by the auto-encoder according to an embodiment of the present invention weights the matrix by focusing on important parameters while reducing the influence of ambiguous parameters and compresses the resulting bottleneck feature, and thus is effective in detecting the autism spectrum in infants and young children.

The deep learning-based method for predicting autism spectrum disorder in infants and young children described above can be implemented as computer-readable code on a computer-readable medium. The computer-readable recording medium may be, for example, a removable recording medium (CD, DVD, Blu-ray disk, USB storage device, removable hard disk) or a fixed recording medium (ROM, RAM, computer-equipped hard disk). The computer program recorded on the computer-readable recording medium may be transmitted to other computing device through a network such as the Internet so that the program can be installed and used on the other computing device.

In the above, even though all the components constituting the embodiments of the present invention are described as being combined or operating in combination, the present invention is not necessarily limited to these embodiments. That is, within the scope of the purpose of the present invention, all of the components may be operated by selectively combining one or more of them.

Although operations are shown in the drawings in a specific order, it should not be understood that the operations must be performed in the specific order shown or sequential order or that all illustrated operations must be performed to obtain the desired results. In certain situations, multitasking and parallel processing may be advantageous. Moreover, the separation of the various components in the embodiments described above should not be construed as necessarily requiring such separation, and it must be understood that the program components and systems described may generally be integrated together into a single software product or packaged into multiple software products.

So far, the present invention has been discussed based on its embodiments. A person skilled in the art to which the present invention pertains will understand that the present invention may be implemented in a modified form without departing from the essential characteristics of the present invention. Therefore, the disclosed embodiments should be considered from an illustrative rather than a restrictive perspective. The scope of the present invention is set forth in the claims rather than the foregoing description, and all differences within the equivalent scope should be construed as being included in the present invention.

### MODE FOR INVENTION

Mode for Invention has been described together with the above best Mode.

### INDUSTRIAL APPLICABILITY

The present invention can be used as data for diagnosing complex autism spectrum disorder by increasing the accuracy of autism spectrum disorder prediction by using the voices of children with autism spectrum disorder (ASD), which are distinct from children with typical development (TD). Therefore, the present invention has industrial applicability.

1. A deep learning-based device for predicting autism spectrum disorder in infants and young children, comprising:
an input unit for inputting segmented speech data;
a first extraction unit for extracting speech features for classification of autism spectrum disorder (ASD);
a second extraction unit for extracting auto-encoder-based speech features; and
a classification unit for classifying the autism spectrum disorder using the speech features.

2. The device according to claim 1, wherein the first extraction unit extracts eGeMAPS features.

3. The device according to claim 1, wherein the second extraction unit reconstructs the speech features using the speech features extracted by the first extraction unit as input value.

4. The device according to claim 1, wherein the device constructs a joint optimization model using an auto-encoder and a deep learning-based classifier model.

5. A deep learning-based method for predicting autism spectrum disorder in infants and young children, wherein the method is performed by a deep learning-based device for predicting autism spectrum disorder in infants and young children, comprising the steps of:
receiving and segmenting speech data;
extracting speech features from the speech data;
embedding values of the features using an auto-encoder; and
classifying an autism spectrum disorder.

6. The method according to claim 5, wherein the step of extracting speech features from the speech data includes extracting eGeMAPS features.

7. The method according to claim 5, wherein the step of embedding values of the features using an auto-encoder includes reconstructing and extracting speech features using an auto-encoder.

8. The method according to claim 5, wherein the method constructs a joint optimization model using an auto-encoder and a deep learning-based classifier model.

9. A computer program recorded on a computer-readable recording medium which executes the method according to claim 5.
